(19) United States
(12) Patent Application Publication
(10) Pub. No.: US 2010/0198249 A1
(43) Pub. Date: Aug. 5, 2010

(54) DEVICE FOR INFLATING A SURGICAL IMPLANT

(75) Inventor: Andre Claude Sabliere, Saint Etienne (FR)

Correspondence Address: DALY, CROWLEY, MOFFORD & DURKEE, LLP, SUITE 301A, 354A TURNPIKE STREET, CANTON, MA, 02021-2714, US

(73) Assignee: European Company of Study and Research for Devices for the Implantaion by Laparoscopy, Vienna (FR)

(21) Appl. No.: 12/738,209

(22) PCT Filed: Oct. 16, 2008
(86) PCT No.: PCT/FR08/01452

§ 371 Date: Apr. 15, 2010

(30) Foreign Application Priority Data
Oct. 16, 2007 (FR) 0707256

(51) Int. Cl. A61M 29/00

(52) U.S. Cl. 606/192

(57) ABSTRACT

The invention relates to an inflation device (1) for inflating an expansible surgical implant (2), wherein said implant (2) is to be positioned inside the body of a human or an animal and is capable of changing, by supplying a filling fluid, from a deflated configuration to an inflated configuration in which it assumes a predetermined volume, said inflation device (1) being characterised in that it comprises a cartridge (3) adapted to be connected with the implant (2) for inflating the same, said cartridge (3) containing a predetermined amount of inflating fluid that does not exceed the amount of inflating fluid to be contained in the implant (2) for the latter to reach its predetermined volume, and in that it comprises an inflation end piece (4) including a means for adjusting the flow (5) of the filling fluid fed into the implant (2). The invention can be used in devices for inflating surgical implants.

### TECHNICAL FIELD

The present invention relates to the general technical field of devices allowing the modification of the configuration of surgical implants in order to allow their functional shaping, said surgical implants being designed to be inserted into the human or animal body.

The present invention relates in particular to the general technical field of devices allowing the inflation, by the addition of an inflation fluid, of surgical implants designed to be inserted into the human or animal body.

The present invention relates to an inflation device designed to inflate an expandable surgical implant, said implant being designed to be positioned inside a human or animal body and being capable of passing, by the addition of inflation fluid, from a deflated configuration to an inflated configuration in which it has a predetermined volume.

The present invention also relates to a system comprising:
an expandable surgical implant designed to be positioned inside a human or animal body, said implant being capable of passing, by the addition of an inflation fluid, from a deflated configuration to an inflated configuration in which it has a predetermined volume,
and an inflation device designed to inflate said surgical implant.

It is known practice to insert surgical implants into the human body in a deflated or flattened configuration and to shape them after their insertion into the body of the patient. This technique is used in particular for shaping intra-gastric balloons designed for the treatment of obesity and positioned for this purpose in the stomach of a patient in order to help him to slim down. Once it has reached its functional shape, such an intra-gastric balloon occupies a considerable space in the stomach of the patient which is normally occupied by ingested food.

To bring the intra-gastric balloon to its functional shape after it has been positioned in a deflated configuration in the stomach, it is known practice to fill the intra-gastric balloon with an inflation fluid, which, for example, is made of air. The air is inserted into the balloon with the aid of a commercially-available manual pump furnished with an inlet orifice, provided with a nonreturn valve, through which the pump first sucks the ambient air and then transfers it into the balloon.

Such a technique, although it provides the possibility of inflating the intra-gastric balloon after it has been installed in the stomach and of allowing it to acquire its functional shape, nevertheless suffers from drawbacks that are not negligible.

The inflation of an intra-gastric balloon with air by means of a manual pump first of all does not allow the balloon to be precisely filled. The practitioner is actually forced to repeat several times the operation of sucking and transferring air with the aid of the manual pump in order to manage the complete filling of the balloon. The repetition of these operations does not allow the practitioner to have an accurate assessment of the exact quantity of air already injected into the balloon, which contributes to the inaccuracy and the slowness of this technique.

The inflation of the balloon makes a decisive contribution to therapeutic effectiveness and to the comfort of the patient. It is possible for the practitioner, in addition to the inaccuracy of inflation caused by the use of a manual pump, to vary, in a manner which may or may not be intentional and controlled, the quantity of air injected. The use of the manual pump therefore has two major drawbacks in terms of use by the practitioner. The practitioner may specifically on the one hand unintentionally inject an incorrect quantity of air into the intra-gastric balloon or, on the other hand, intentionally choose not to follow the inflation recommendations and to inject a quantity that differs from that recommended. In both cases, the variations and inaccuracies may lead to problems of underinflation or of overinflation of the intra-gastric balloon.

Underinflation of the intra-gastric balloon may lead to loss of therapeutic effectiveness (insufficient weight loss) or to the risk of seeing the balloon move toward the intestine. In the case of an overinflated balloon, the patient may suffer nausea, epigastric pain and even ulcers. Moreover, overinflation of a balloon causes the stretching of the walls forming it and therefore a weakening of the strength of its walls with respect to the air seal and the aggressive environment formed by the stomach. In other words, an overinflated balloon may have harmful or even dangerous consequences for the patient and deteriorates rapidly.

Moreover, the intra-gastric balloons of the prior art that can be inflated with air have a limited period of use in the body of the patient, notably because of the intrinsic porosity to air of the materials used to manufacture the walls of these intra-gastric balloons. Specifically, the materials forming the walls are in particular of flexible elastomer, notably silicone or polyurethane. Although having a valuable elasticity and a resistance to the aggressive environment inside the stomach, these materials are usually porous to air.

This porosity may cause the intra-gastric balloon to deflate and even to migrate toward the intestine. These air-inflated intra-gastric balloons may therefore require replacement or removal from the stomach of the patient before the end of the therapeutic treatment.

To remedy these problems of porosity of the wall of the intra-gastric balloon to air, it is well known to use techniques of surface-treatment of the walls, in particular techniques of covering said walls with protective agents. However, this technique contributes to increasing the complexity of manufacture of the intra-gastric balloon and consequently to greatly increasing the price thereof.

### SUMMARY OF THE INVENTION

The objects assigned to the invention are consequently aimed at providing a remedy for the various drawbacks listed above and at proposing a new inflation device allowing an inflation, that is simultaneously safe, precise and effective, of an expandable surgical implant, preferably an intra-gastric balloon.

Another object of the invention is to propose a new inflation device leading to a precise, defined and one-shot inflation of the implant.

Another object of the invention is to propose a new inflation device causing a rapid inflation of the implant.

Another object of the invention is to propose a new inflation device that is materially economical and easy to put in place.

Another object of the invention is to propose a new inflation device which is simple and logical to use.

Another object of the invention is to propose a new inflation device operating independently and rapidly.

Another object of the invention is to propose a new inflation device having a protective use and a good seal.

Another object of the invention is to propose a new inflation device that can be simply and securely connected to the implant.

Another object of the invention is to propose a new inflation device allowing the user to effectively control the flow rate of fluid intended to inflate the surgical implant.

Another object of the invention is to propose a new inflation device allowing an effective and secure inflation speed.

Another object of the invention is to propose a new inflation device making it possible to prevent the deflation of the implant during the inflation operation.

Another object of the invention is to propose a new inflation device preventing any risk of overinflation of the surgical implant.

Another object of the invention is to propose a new inflation device making information on the inflation parameters during inflation accessible to the user.

Another object of the invention is to propose a new inflation device promoting a period of use of the surgical implant that matches the period of therapeutic treatment.

Another object of the invention is to propose a new inflation system of a surgical implant allowing a secure, precise and effective inflation of the implant while being easy to use.

The objects assigned to the invention are achieved with the aid of an inflation device designed to inflate an expandable surgical implant, said implant being designed to be positioned inside a human or animal body and being capable of passing, by the addition of inflation fluid, from a deflated configuration to an inflated configuration in which it has a predetermined volume, said inflation device being characterized in that it comprises a cartridge designed to be connected to the implant in order to carry out the inflation of the latter, said cartridge containing a predetermined quantity of inflation fluid which does not exceed the quantity of inflation fluid that the implant is to contain in order to reach its predetermined volume, and in that it comprises an inflation end piece comprising a means for adjusting the flow rate of the inflation fluid travelling into the implant.

The objects assigned to the invention are also achieved with the aid of a system comprising:
an expandable surgical implant designed to be positioned inside a human or animal body, said implant being capable of passing, by the addition of inflation fluid, from a deflated configuration to an inflated configuration in which it has a predetermined volume,
and an inflation device designed to inflate said surgical implant,
   characterized in that the inflation device comprises a cartridge designed to be connected to the implant in order to inflate the latter, said cartridge containing a predetermined quantity of inflation fluid which does not exceed the quantity of inflation fluid that the implant is to contain in order to reach its predetermined volume, and in that said inflation device comprises an inflation end piece comprising a means for adjusting the flow rate of the inflation fluid travelling into the implant.

### BRIEF DESCRIPTION OF THE DRAWINGS

Other objects and advantages of the invention will be more clearly apparent on reading the following description and with the aid of the appended drawings given purely as an illustration and not being limiting, in which:

FIG. 1 illustrates, in an exploded schematic view, a preferred embodiment of the system of the invention in which the expandable surgical implant is an intra-gastric balloon.

FIG. 2 illustrates, in an exploded schematic view, a detail of the system of the invention of FIG. 1.

FIG. 3 illustrates, in a schematic view, a detail of the system of the invention of FIG. 1.

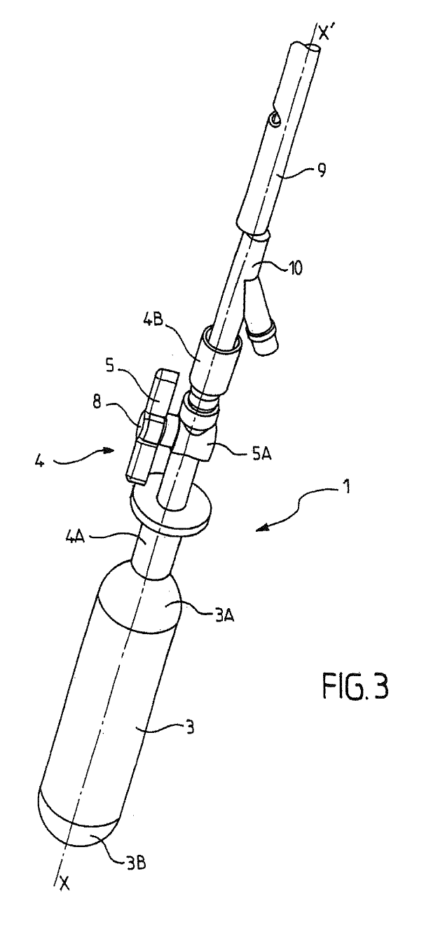

FIG. 4 illustrates, in a view in longitudinal section, a detail of the system of the invention of FIG. 1.

### BEST MANNER OF EMBODYING THE INVENTION

The inflation device 1 of the invention is designed to inflate an expandable surgical implant 2.

Within the meaning of the invention, an expandable surgical implant 2 is an implant that can withstand a deformation or a modification of its spatial configuration before or after it is inserted into the human or animal body. The expandable surgical implant 2 of the invention is designed to be positioned inside a human or animal body.

Advantageously, the expandable surgical implant 2 as defined in the present invention is an expandable intra-gastric balloon designed for the treatment of obesity. The intra-gastric balloon is consequently designed to be preferably implanted into the stomach of a patient as part of a treatment for obesity. Preferably, the intra-gastric balloon is inserted into the stomach of a patient via natural channels, preferably the esophageal channels. Advantageously, the intra-gastric balloon is first of all inserted via the mouth of the patient and then progresses into the esophagus in order subsequently to reach the stomach.

The following description and the figures that illustrate it preferably relate to an intra-gastric balloon designed to be inserted into the stomach of a patient in order to treat obesity. Any other expandable surgical implant according to the foregoing, for example a mammary implant, is quite evidently able to be envisaged, nevertheless without departing from the context of this invention.

The expandable surgical implant 2 comprises one or more walls preferably made of a material that is sufficiently flexible to allow said implant 2 to pass easily from a deflated configuration to an inflated configuration. Preferably, the implant 2 has one or more walls made of flexible elastomer, for example of silicone. Naturally, the material forming said wall may advantageously be any other material satisfying the criteria of elasticity, of compatibility with the human body and of resistance to the surrounding medium, notably the particularly aggressive stomach medium.

The expandable surgical implant 2 is capable of passing from a deflated configuration to an inflated configuration. In other words, the expandable surgical implant 2 is presented initially in a deflated configuration, flattened and of low volume, in which the expandable surgical implant 2 is not functional. The expandable surgical implant 2 then passes, by the addition of inflation fluid, to an inflated configuration in which it has a predetermined volume.

Within the meaning of the invention, an inflated configuration is a functional configuration which makes reference on the one hand to the volume and on the other hand to the shape that are conferred on the expandable surgical implant 2 in order to provide it with the therapeutic capability that is expected in the context of a treatment for obesity.

The inflation device 1 comprises a cartridge 3 designed to be connected to the implant 2 in order to inflate the latter. The cartridge 3 contains a predetermined quantity of inflation fluid which does not exceed the quantity of inflation fluid that the implant is to contain in order to reach its predetermined volume, namely its optimal volume for a therapeutic treatment. In other words, the cartridge 3 is designed to inflate the implant 2 until the latter reaches its predetermined volume, that is to say its functional volume that allows it to have a therapeutic effectiveness within the body.

The cartridge 3 is preferably a receptacle that can contain an inflation fluid used to inflate the expandable surgical implant 2. Advantageously, the cartridge 3 has an elongated shape on an axis of extension (XX′) that can be seen in the various figures. Preferably, the cartridge 3 has one end 3A close to the implant 2 and one end 3B at a distance from the implant 2, said ends 3A and 3B each having a substantially rounded shape.

Advantageously, the cartridge 3 is substantially nondeformable, that is to say that it is difficult or even impossible to modify its shape. The cartridge 3 is therefore preferably rigid, unbreakable, and has an identical shape irrespective of its use. In a particularly advantageous manner, the cartridge 3 is made of a material that is capable of conferring on said cartridge 3 a sufficient rigidity to prevent its deformation during its storage and its use. Preferably, the cartridge 3 is also sealed against the inflation fluid that it contains, that is to say that it is manufactured with one or more materials having a seal against the inflation fluid.

Therefore the material forming said cartridge 3 is preferably a material that makes it possible to manufacture a cartridge 3 that is simultaneously rigid, nondeformable and sealed against the inflation fluid. This material is advantageously a metal, for example stainless steel. It is however perfectly envisageable that the cartridge 3 is made of a solid plastic or of any other material satisfying the criteria of rigidity, nondeformability, and sealing mentioned above.

The cartridge 3 advantageously contains a quantity of inflation fluid that does not exceed the quantity of inflation fluid necessary to reach the functional inflated configuration of the expandable surgical implant 2.

Preferably, the cartridge 3 contains a predetermined quantity of inflation fluid substantially equal to the quantity of inflation fluid that the implant 2 is to contain to reach its predetermined volume. The cartridge 3 is filled in construction with a precise quantity of inflation fluid, the filling being carried out preferably before the operation of inflating the implant 2 during the process of manufacturing the cartridge 3, for example in a factory. In other words, the cartridge 3 contains, prior to inflation, a predetermined, unmodifiable quantity after manufacture, of inflation fluid, that is to say that the cartridge 3 contains a single dose of fluid retained in a sealed manner inside it.

For this purpose, the cartridge 3 preferably comprises a sealing membrane 3D closing off in a sealed manner a single aperture 3C of said cartridge 3. In a preferred manner, the sealing membrane 3D is made of flexible material, sealed against the pressurized fluid contained in the cartridge 3, said sealing membrane 3D being for example made of plastic material.

Advantageously, the intra-gastric balloon inflated with its predetermined volume contains a quantity of inflation fluid of between 200 and 800 cm³, preferably between 500 and 700 cm³, particularly preferably substantially equal to 700 cm³. Consequently, the cartridge 3 advantageously contains a quantity of fluid of between 200 and 800 cm³, preferably of between 500 and 700 cm³, particularly preferably substantially equal to 700 cm³.

In a preferred manner, the cartridge 3 contains a quantity of inflation fluid that cannot exceed the predetermined volume of the implant 2. This technical measure makes it possible to ensure security of functional shaping of the implant 2 in the body of the patient. Specifically, with the cartridge 3, it is not possible to exceed the maximum inflation volume of the implant 2 and to risk an overinflation of the implant 2.

Preferably, the quantity of inflation fluid included in the cartridge 3 may not allow the implant 2 to be overinflated. Therefore, a user, for example a practitioner, can inflate the implant 2, preferably an intra-gastric balloon, in a single shot with the aid of the cartridge 3 without risking overinflating, that is to say inflating to excess, the intra-gastric balloon. This technical measure makes it possible to prevent all the inconveniences and risks that exist for the patient in the case of inflation with a manual pump. The use of the cartridge 3 also makes it possible to obtain an even and precise inflation of the implant 2 in order to obtain the exact predetermined volume desired by the practitioner and/or recommended by the instructions of use.

The inflation device 1 of the invention advantageously comprises a means for propelling the inflation fluid contained in the cartridge 3 out of the cartridge 3 in order to inflate the implant 2. Preferably, the inflation fluid can be propelled, that is to say expelled, out of the cartridge 3 with the aid of a propulsion means. The inflation fluid is then preferably expelled, ejected, from the cartridge 3 in the direction of the implant 2 in order to allow the inflation of said implant 2.

Within the meaning of the invention, a propulsion means corresponds preferably to any means making it possible to cause the inflation fluid contained in said cartridge 3 to exit in the direction of the implant 2 in order to inflate it.

Advantageously, the means for propelling the inflation fluid contained in the cartridge 3 is loaded into said cartridge 3. In other words, the propulsion means advantageously forms an integral part of the cartridge and is therefore not an element external to the latter. In a preferred embodiment of the present invention, the propulsion means is a pressurization of the inflation fluid in the hermetically closed cartridge 3, the opening of the cartridge 3, notably by piercing of the sealing membrane 3D, causing the inflation fluid to automatically leave the cartridge 3 under the effect of the pressure prevailing in the cartridge 3.

Therefore, preferably, the inflation fluid contained in the cartridge 3 is a pressurized fluid capable of being self-propelled from the cartridge 3 in the direction of the implant 2 in order to inflate the latter when the cartridge 3 is connected to the implant 2. Advantageously, when the cartridge 3 is connected or hooked up to the implant 2, the inflation fluid contained, enclosed, in said cartridge 3 is expelled outward from said cartridge 3 with the aid of the propulsion means.

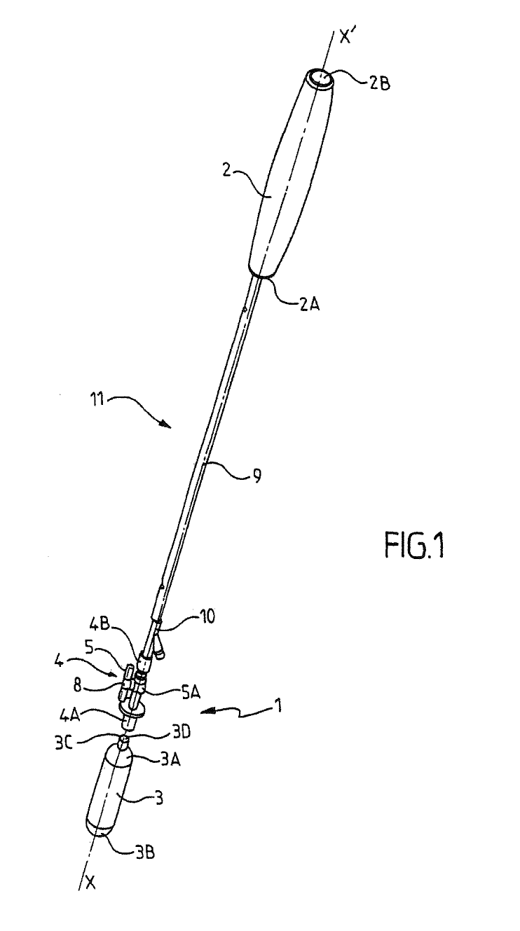

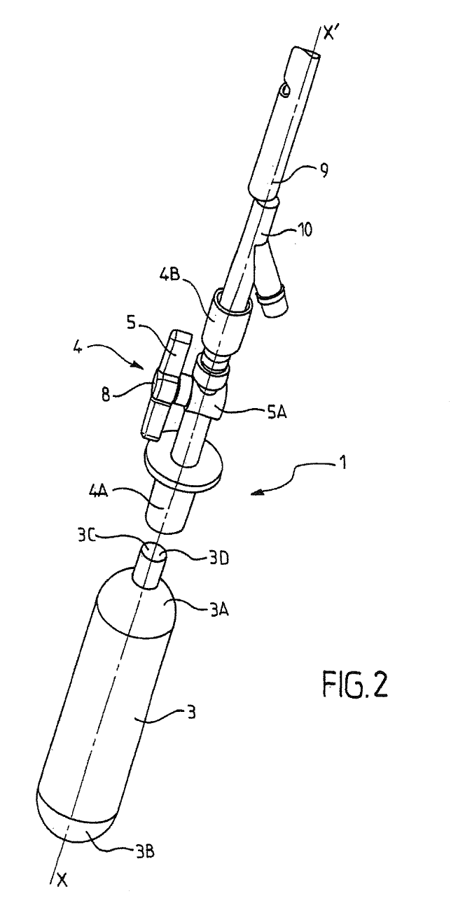

Preferably, the cartridge 3 comprises a single aperture 3C allowing the propulsion of the inflation fluid in a single direction toward the implant 2 _{w}hen ₜhe cartridge 3 is connected to the implant 2. As illustrated in FIGS. 1, 2 and 4, the aperture 3C is situated at the end 3A of the cartridge 3, namely on the side of the implant 2.

Within the meaning of the present invention, the connection, that is to say the joining, of the cartridge 3 at its aperture 3C, to the implant 2 advantageously allows the automatic expulsion of the inflation fluid because of the pressurization of said inflation fluid in the cartridge 3, said pressurization preferably being carried out when the cartridge 3 is filled. Preferably, the expulsion of the inflation fluid from the cartridge 3 to the implant 2 is carried out in a sealed manner, that is to say that said cartridge 3 is connected to the implant 2 so as to prevent any leakage of inflation fluid to the outside of the cartridge 3 and of the implant 2. Preferably, the connection of the cartridge 3 to the implant 2, for example by piercing of a membrane, thus preferably causes the cartridge 3 to be placed in fluidic communication with the implant 2.

The expulsion of the fluid is preferably carried out in one shot, the whole of the content of the cartridge 3 being able to be used for the optimal inflation of the implant 2. The self propulsion of the fluid from the cartridge 3, from the latter and in the direction of the implant 2, has the value of achieving an inflation in a single and continuous inflation step, said inflation being rapid, precise and requiring no external intervention or complicated manipulation of the practitioner. Specifically, for this type of inflation with the cartridge 3, no manual pump or any external force is necessary.

Preferably, the sealing membrane 3D closes the aperture 3C of said cartridge 3 in a sealed manner at the zone of connection of said cartridge 3 to the implant 2, namely at the end 3A of said cartridge 3.

Preferably, the inflation device 1 of the invention comprises an inflation end piece 4 extending between two ends, the first 4A being designed to be connected to the cartridge 3 and the second 4B to the implant 2, said inflation end piece 4 comprising, at its first end 4A, a means 4C for piercing the sealing membrane 3D when said cartridge 3 is connected to the inflation end piece 4.

Advantageously, the cartridge 3 is not connected directly to the implant 2, the inflation end piece 4 serving as an intermediary between said cartridge 3 and the implant 2, said inflation end piece 4 establishing a sealed connection between the cartridge 3 and the implant 2, said sealed connection making it possible to prevent any leakage of inflation fluid to the outside of the assembly formed by the cartridge 3, the inflation end piece 4 and the implant 2. Preferably, this inflation end piece 4 makes the operation of inflating the implant 2 easier.

The means 4C making it possible to pierce the sealing membrane 3D is preferably a pointed end terminating the central channel 4D at the end 4A of the inflation end piece 4, said channel 4D traversing longitudinally on the axis of extension (XX′) the inflation end piece 4 and making it possible to maintain the continuity of the flow of inflation fluid between the cartridge 3 and the implant 2. In other words, the piercing means 4C preferably corresponds to any means making it possible to easily pierce the sealing membrane 3D which closes off the aperture 3C of the cartridge 3. During the connection of the cartridge 3 to the inflation end piece 4 at the means 4C, the sealing membrane 3D is pierced by the means 4C, thus causing the expulsion of the pressurized fluid contained in said cartridge 3 in the direction of the implant 2 through the inflation end piece 4 via the channel 4D, the path of the inflation fluid from the cartridge 3 to the implant 2 having no leaks to the outside.

Preferably, the inflation end piece 4 comprises a means for adjusting the flow rate 5 of the inflation fluid travelling into the implant 2. Advantageously, the user of the inflation device 1 can adjust the flow rate of the fluid travelling to the implant 2 in order to inflate the latter, the user thus being able for example to accelerate or slow down the flow of fluid leaving the cartridge 3.

In a preferred manner, the means 5 for adjusting the flow rate of the inflation fluid is a flow-rate throttle 5A calibrated so that the flow rate of the inflation fluid does not exceed a threshold value, advantageously of between 50 and 500 cm³ per minute, preferably of between 100 and 300 cm³ per minute, and yet more preferably substantially equal to 300 cm³ per minute. The flow-rate throttle 5A corresponds substantially to a tap the operation of which by the user preferably makes it possible to vary the flow rate of the fluid in question.

The flow rate of fluid allowed by the flow-rate throttle 5A is preferably first calibrated in order to avoid having too great a flow rate that can damage the implant 2, notably by stretching its wall, or leading to too rapid an inflation of said implant 2. Advantageously, irrespective of the position of the flow-rate throttle 5A given by the practitioner, the flow rate of the inflation fluid cannot exceed a threshold value substantially equal to 300 cm³ per minute.

The means 5 for adjusting the flow rate is particularly suited and functional for the filling of an intra-gastric balloon, the latter having to be inflated in a contained, secure and controlled manner in order to prevent any too rapid, sudden or violent inflation likely to damage the biological tissues, notably the wall of the stomach. Such too sudden inflation can for example cause lesions leading to pains perceptible by the patient and being able to lead, in certain cases, to the withdrawal of the balloon. Moreover, the means 5 for adjusting the flow rate makes it possible to ensure a harmonious inflation of the intra-gastric balloons comprising several pockets. For this type of balloon, the means 5 for adjusting the flow rate, notably the flow-rate throttle 5A, promotes an adjusted, balanced and even filling of the internal pocket of the balloon so as to obtain a nontraumatic inflation.

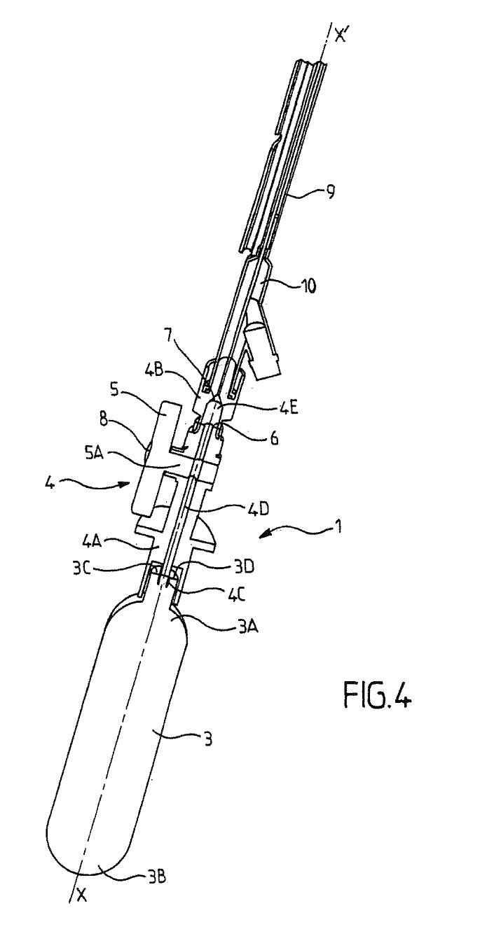

Advantageously, as illustrated in FIG. 4, the inflation end piece 4 also comprises a nonreturn valve 6 in order to prevent the implant 2 from deflating when the cartridge 3 is disconnected from the inflation end piece 4. The nonreturn valve 6 is positioned advantageously at the inflation end piece 4 and makes it possible to prevent any deflation of the implant 2 when the cartridge 3 is disconnected from the inflation end piece 4 whether intentionally or not.

Moreover, the inflation end piece 4 preferably comprises a fusible membrane 7 which is automatically pierced in order to allow an immediate self-deflation of the implant 2 when the pressure in the implant 2 exceeds a threshold value, preferably between 0.3 and 0.7 bar, advantageously substantially equal to 0.5 bar. Preferably, the fusible membrane 7 is positioned just downstream of the flow-rate throttle 5 in the direction of flow of the inflation fluid so as to be in direct contact with the internal pressure of the implant 2. Advantageously, the fusible membrane is close to the end 4B of the inflation end piece 4, more particularly right next to a bottleneck 4E at which the diameter of the channel 4D is substantially reduced.

Preferably, this fusible membrane 7 is a safety means making it possible to very rapidly discharge the inflation fluid from the implant 2 if the pressure prevailing within the implant exceeds a threshold value, previously defined notably according to the type of implant 2 used and the predetermined volume of the implant in the inflated configuration. If the pressure is abnormally high and harmful for the implant 2 and therefore for the patient, the fusible membrane 7 is pierced and allows the immediate self-deflation of the implant 2, in particular of the intra-gastric balloon, in a few seconds, that is to say the automatic and instantaneous leakage of the inflation fluid from the implant 2.

Moreover, the inflation end piece 4 preferably comprises a means 8 for reading the pressure prevailing within the implant 2. This means 8 for reading the pressure is preferably positioned on the flow-rate throttle 5A so as to be easily visible to the practitioner during the operation of inflating the implant 2. The means 8 for reading the pressure is preferably a manometer indicating simply the pressure in the implant. By virtue of this means 8 for reading the pressure, the practitioner can monitor in real time the pressure that is present in the implant and can therefore modulate the flow rate of inflation fluid with the aid of the flow-rate throttle 5A.

Advantageously, the inflation end piece 4 is connected to the implant 2 via a duct 9, for example a catheter 9, so that the cartridge 3 and the inflation end piece 4 are situated outside the body during the whole inflation operation. After the functional inflation of the implant 2, the catheter 9 is removed from the body. Thus, the implant 2 of the invention advantageously plays a therapeutic role as such and is not designed to be permanently connected to the outside, and in particular to the cartridge 3, after it has been inflated.

Advantageously, the inflation fluid used to inflate the implant 2 is a gas. The choice is therefore preferably to fill the implant 2, in particular an intra-gastric balloon, with the aid of a gas.

Expandable surgical implants inflated with air, in particular intra-gastric balloons, have walls routinely made of flexible elastomer notably of silicone or of polyurethane. Elastomer, because of its porosity to small molecules, such as the dioxygen of the air for example, has a variable or even rather weak seal with respect to the air contained in the balloon. Therefore, these intra-gastric balloons inflated with air have a limited period of use in the body of the patient, notably because of the porosity to the air of the elastomer present in the walls of these balloons.

There are other gases that could, because of the size of their molecules and in particular because of their molar mass greater than that of oxygen, have slow diffusion speeds through the walls of the intra-gastric balloons. These are in particular carbon dioxide or nitrogen. However, these gases are not used to inflate intra-gastric balloons because they are difficult to handle in the context of a surgical implantation of an intra-gastric balloon in a hospital environment.

Specifically, they are more often packaged in containers that are not very practical to use and require special equipment and connections very markedly complicating the operation of implanting an intra-gastric balloon.

The cartridge 3 of the invention makes it possible to remedy the drawbacks of the inflation devices of the prior art by making it possible to use any type of gas for the inflation of an expandable surgical implant 2, notably an intra-gastric balloon. Specifically, the use of a cartridge containing a pressurized gas in a predetermined and precise quantity for a secure and effective inflation preferably allows an inflation of the implant 2 in a simple and particularly secure manner for the implant 2 and the patient. Therefore, with the cartridge 3, which is preferably both rigid, undeformable, unbreakable and made of a material sealed against the gas, according to the foregoing, it can be envisaged to use a pressurized gas to inflate, in a secure and reliable manner, the implant 2.

Preferably, the inflation gas used in the context of the present invention is therefore a gas with a molar mass greater than that of oxygen, in a particularly preferred manner carbon dioxide or nitrogen.

By virtue of the presence of a sealed cartridge 3 making it possible to pressurize an inflation gas and being able to be used in a secure and simple manner, it is possible to use preferably a variety of inflation gases and not only air. Any type of gas, that is not toxic for the patient and satisfies the criterion of low diffusion through the wall of the implant 2, can therefore preferably be used for inflating said implant 2. Therefore, it can also be envisaged that the cartridge 3 advantageously contains a rare gas, for example argon.

Moreover, the invention relates, as such and independently of all the features previously described, to an implant inflated with a gas other than air. The expandable surgical implant 2 designed to be positioned inside a human or animal body is capable of passing, by the addition of inflation fluid, from a deflated configuration to an inflated configuration in which it has a predetermined volume, said expandable surgical implant being characterized in that the inflation fluid is a gas consisting of molecules having a molar mass substantially greater than that of dioxygen.

Preferably, the expandable surgical implant 2 is an intra-gastric balloon as defined in the foregoing.

The present invention also relates to a system 11 comprising:
an expandable surgical implant 2 designed to be positioned inside a human or animal body, said implant 2 being capable of passing, by the addition of inflation fluid, from a deflated configuration to an inflated configuration in which it has a predetermined volume,
and an inflation device 1 designed to inflate said surgical implant 2,
   characterized in that the inflation device 1 comprises a cartridge 3 designed to be connected to the implant 2 in order to inflate the latter, said cartridge 3 containing a predetermined quantity of inflation fluid which does not exceed the quantity of inflation fluid that the implant 2 is to contain in order to reach its predetermined volume, and in that said inflation device 1 comprises an inflation end piece 4 comprising a means 5 for adjusting the flow rate of the inflation fluid travelling into the implant 2. Preferably, the adjustment means 5 makes it possible to adjust the flow rate of the fluid when it leaves the cartridge 3, that is to say from the inflation device 1, in the direction of the implant 2.

Within the meaning of the invention, said system 11 comprises an expandable surgical implant 2 as defined above and corresponding preferably to an expandable intra-gastric balloon designed for the treatment of obesity. The balloon is advantageously designed to be positioned in the body and, on its own, to be therapeutically effective throughout the period of treatment. The system 11 moreover comprises an inflation device 1 as has been described in the foregoing.

Moreover, the system 11 also comprises preferably, as illustrated in FIG. 1, a duct 9 connecting the inflation end piece 4 to the implant 2. In other words, in a preferred embodiment of the invention, the system 11 comprises simultaneously the inflation cartridge 3, the inflation end piece 4, the duct 9 and the implant 2, the system 11 being completely sealed against the inflation fluid.

Advantageously, the duct 9 is made of flexible material, sealed against the inflation fluid and not traumatic for the patient. Preferably, the duct 9 is a silicone tube, for example a catheter 9, with a length allowing the inflation device 1 to be positioned outside the body of the patient in order to make its use easier for the practitioner.

Optionally, the system 11 comprises an intermediate, Y-shaped part 10 which connects the inflation end piece 4 to the duct 9. This intermediate part 10 is preferably used for the addition of other substances, if necessary, to the inside of the intra-gastric balloon.

Said system 11 makes it possible to inflate, in a simple, precise and safe manner, an expandable surgical implant, in particular an intra-gastric balloon. The present invention has the advantage of being able to fill the implant with any gas, notably with gases which diffuse slowly or even in a virtually zero manner through the wall of the implant.

The system 11 of the invention also makes it possible to precisely inflate the implant for the purpose of obtaining the predetermined volume of said implant, by eliminating any risk of underinflation or of overinflation.

The present invention also relates to a method for inserting an intra-gastric balloon into the body of a patient. When the intra-gastric balloon is placed in the body of the patient, the practitioner uses the system 11 as detailed above. Preferably, the intra-gastric balloon is presented in a deflated form, folded in the form of an “ovoid umbrella”. The intra-gastric balloon is connected to a silicone duct 9 at its end 2A, said duct 9 itself being connected, outside the patient, to the inflation end piece 5.

The deflated intra-gastric balloon is preferably inserted, via the natural channels, notably via the esophagus, into the stomach of the patient. The balloon is first of all inserted through the mouth of the patient, then it progresses into the esophagus in order finally to reach the stomach of the patient. The practitioner then connects the cartridge 3 containing the inflation gas to the inflation end piece 4. The pointed end piece corresponding to the piercing means 4C present on the inflation end piece 4 at its end 4A pierces the sealing membrane 3D closing off the cartridge 3 and allows the pressurized gas contained in the cartridge 3 to be propelled out of the cartridge 3 toward the inflation end piece 4.

The gas passes through the end piece 4 in the channel 4D in order to fill the intra-gastric balloon. The seal against the inflation gas of the various connections established between the cartridge 3, the inflation end piece 4, the duct 9 and the implant 2 makes it possible to prevent any leakage of the inflation gas out of the whole of the system 11. The practitioner can at any time monitor the pressure present in the implant 2 with the aid of the manometer 8 placed on the inflation end piece 4. Preferably, the practitioner uses the means 5 for adjusting the flow rate in order to modify, at the outlet of the cartridge 3, the speed and the quantity of the gas intended to fill the balloon so as to prevent any too sudden inflation and/or too rapid inflation of the balloon which could be particularly dangerous for the patient. The practitioner can therefore vary the flow rate of inflation gas with the aid for example of the flow-rate throttle 5A.

In other words, the adjustment means 5, comprising notably a flow-rate throttle 5A, makes it possible to provide a harmonious, precise and even inflation of the implant, so as to limit any risk of too violent or too sudden an inflation that can seriously damage the tissues of the stomach. Moreover, this controlled inflation of the balloon, ensured by the flow-rate adjustment means 5, is perfectly suited to the filling of intra-gastric balloons comprising several pockets or walls. Specifically, for the multi-pocketed or multi-walled balloons, the means 5 for adjusting the flow rate ensures a safe inflation, protective of the balloon and of the patient, controlled and reliable.

Since the cartridge 3 contains precisely the quantity of gas necessary for the functional inflation of the intra-gastric balloon, the practitioner cannot cause any overinflation of the balloon. This technical measure makes it possible to ensure that the practitioner is provided with a safe and precise inflation of the intra-gastric balloon and consequently posing no danger for the patient.

Once the inflation of the intra-gastric balloon is completed, the cartridge 3 is disconnected from the inflation end piece 4, the nonreturn valve 6 making it possible to prevent any unintentional deflation of the balloon. The duct 9 is then removed from the body of the patient.

The inflation device 1 containing both the cartridge 3 and the inflation end piece 4 is preferably for a single use.

Alternatively, the cartridge 3 can be reused and again be filled with a pressurized gas for a further subsequent use. Similarly, the inflation end piece 4 can advantageously be reused. The cartridge 3 and the end piece 4 are advantageously either recyclable or reusable after a cleaning operation, depending on the standards applicable for the medical devices. These technical measures for reuse of the cartridge 3 and of the inflation end piece 4 make it possible to more rapidly amortize the acquisition costs of this type of equipment and to control the scrappage of waste.

### POSSIBILITY OF INDUSTRIAL APPLICATION

The invention finds its industrial application in the design and manufacture of devices for functionally shaping expandable surgical implants, notably intra-gastric balloons for the treatment of obesity.

1. An inflation device for inflating an expandable surgical implant, the inflation device comprising:
a cartridge designed to be connected to the expandable surgical implant in order to carry out the inflation of the latter, said cartridge containing a predetermined quantity of inflation fluid which does not exceed a quantity of inflation fluid that the expandable surgical implant is to contain in order to reach a predetermined volume
an inflation end piece comprising a means for adjusting the flow rate of the inflation fluid travelling from the cartridge into the expandable surgical implant.

2. The inflation device as claimed in claim 1 wherein the expandable surgical implant is an expandable intra-gastric balloon.

3. The inflation device as claimed in claim 1 wherein the cartridge contains a predetermined quantity of inflation fluid substantially equal to the quantity of inflation fluid that the expandable surgical implant is to contain in order to reach a predetermined volume.

4. The inflation device as claimed in claim 1 further comprising a means for propelling the inflation fluid contained in the cartridge out of the cartridge in order to inflate the expandable surgical implant.

5. The inflation device as claimed in claim 4, wherein the means for propelling the inflation fluid contained in the cartridge is loaded into said cartridge.

6. The inflation device as claimed in claim 4 wherein the cartridge comprises a single aperture allowing the propulsion of the inflation fluid in a single direction toward the expandable surgical implant when the cartridge is connected to the implant.

7. The inflation device as claimed in claim 6, wherein the cartridge comprises a sealing membrane closing off the aperture of said cartridge.

8. The inflation device as claimed in claim 1 wherein the inflation end piece extends between two ends, the first end being designed to be connected to the cartridge and the second end to the implant, said inflation end piece comprising, at its first end, a means for piercing the sealing membrane when said cartridge is connected to the inflation end piece.

9. The inflation device as claimed in claim 1 wherein the means for adjusting the flow rate of the inflation fluid is a flow-rate throttle calibrated so that the flow rate of the inflation fluid does not exceed a threshold value.

10. The inflation device as claimed in claim 1 wherein the inflation end piece comprises a nonreturn valve, which prevents the implant from deflating when the cartridge is disconnected from the inflation end piece.

11. The inflation device as claimed in claim 1 wherein the inflation end piece comprises a fusible membrane which is automatically pierced in order to allow an immediate self-deflation of the implant when the pressure in the implant exceeds a threshold value.

12. The inflation device as claimed in claim 1 wherein the inflation end piece comprises a means for reading the pressure prevailing within the implant.

13. The inflation device as claimed in claim 1 wherein the inflation fluid contained in the cartridge is a pressurized fluid capable of being self-propelled in the direction of the implant in order to inflate the latter when the cartridge is coupled to the implant.

14. The inflation device as claimed in claim 1 wherein the inflation fluid used to inflate the implant is a gas.

15. The inflation device as claimed in claim 1 wherein the cartridge is substantially nondeformable.

16. A system comprising:
an expandable surgical implant configured to be positioned inside a human or animal body, said expandable surgical implant being capable of passing, by the addition of inflation fluid, from a deflated configuration to an inflated configuration in which it has a predetermined volume; and
an inflation device configured to inflate said expandable surgical implant, characterized in that the inflation device comprises a cartridge configured to be connected to the expandable surgical implant in order to inflate the expandable surgical implant, said cartridge containing a predetermined quantity of inflation fluid which does not exceed the quantity of inflation fluid that the expandable surgical implant is to contain in order to reach its predetermined volume, and in that said inflation device comprises an inflation end piece comprising a means for adjusting the flow rate of the inflation fluid travelling into the expandable surgical implant.

17. (canceled)

18. An inflation device for inflating a surgical implant, the inflation device comprising:
a cartridge having walls which define a fluid chamber and an aperture leading thereto with a predetermined quantity of inflation fluid disposed in the fluid chamber;
a sealing membrane disposed over the aperture of said cartridge to provide the fluid chamber as a sealed chamber; and
an inflation end piece coupled to said cartridge and in fluid communication with said sealing membrane, said inflation end piece comprising:
means to pierce said sealing membrane; and
means for adjusting a flow rate of inflation fluid travelling through the aperture of said cartridge.

19. The inflation device of claim 18 wherein said cartridge is provided from a substantially nondeformable material.

20. The device of claim 18 further comprising means for reading pressure within the implant.

21. The device of claim 20 further comprising means for propelling the inflation fluid contained in the cartridge out of the cartridge in order to inflate the expandable surgical implant.

22. The inflation device as claimed in claim 14 wherein the inflation fluid is a gas having a molar mass greater than that of oxygen.
